(19) United States
(12) Patent Application Publication
(10) Pub. No.: US 2019/0239865 A1
(43) Pub. Date: Aug. 8, 2019

(54) Systems and Method for Collecting and Analyzing Tissue Samples from Orthopedic Surgery for use in a Searchable Structured Database for Analysis of Joint Diseases

(71) Applicant: Artios Therapeutics, Inc., Campbell, CA (US)

(72) Inventor: Theodore R. Kucklick, Campbell, CA (US)

(73) Assignee: Artios Therapeutics, Inc., Campbell, CA (US)

(21) Appl. No.: 16/148,978

(22) Filed: Oct. 1, 2018

Related U.S. Application Data
(63) Continuation of application No. 14/990,691, filed on Jan. 7, 2016, now abandoned.
(60) Provisional application No. 62/100,481, filed on Jan. 7, 2015.

(51) Int. Cl. A61B 10/02; A61B 18/14; A61B 10/00

(52) U.S. Cl. CPC A61B 10/0283; A61B 18/149; A61B 2018/00577; A61B 10/0096; A61B 18/148

(57) ABSTRACT

Systems and methods for collecting bio-specimens from arthroscopic or joint surgery, collecting them in a dedicated collection cassette, pre-treating the samples as needed, transporting these samples to a lab, analyzing these samples, and aggregating this data into a searchable and structured dataset that may be analyzed, for correlation and causality of joint diseases and pathology. Tissue may be collected with devices and systems for use in an arthroscopic procedure, an open procedure such as a joint implant, or with a dedicated biopsy device. Bio-specimens include previously discarded blood cartilage, synovium, bone, meniscus and other tissue samples drained from these surgical procedures.

This application is a continuation of U.S. application Ser. No., 14/990,691, filed Jan. 7, 2016, which in turn claims priority to U.S. Provisional Application 62/100,481, filed Jan. 7, 2015.

### FIELD OF THE INVENTIONS

The inventions described below relate to the field of orthopedic and joint surgery such as arthroscopy, arthroplasty, and spine surgery, and the capturing, collection and processing of tissue samples typically discarded as medical waste, analyzing these tissue samples to generate genetic health and disease information, and storing the test results in a searchable database.

### BACKGROUND OF THE INVENTIONS

Joint health is critically important. The cost of joint diseases in the United States is estimated at over 450 billion dollars per year (AAOS 2004, based on trend). This is an estimated 5 trillion dollar global problem. Arthritis alone is an estimated 1.2 trillion dollar problem. Currently no method exists to collect consistent population scale data on joint health and disease. The only data that is recorded often is limited to billing and coding information, which only catalogs the procedures done. Little if any in depth data about a patient's joint health is recorded.

The discovery and identification of the exact causes of joint disease are very difficult to uncover using conventional means. Scientific studies are often done on narrow problems, or on non-human subjects such as mice. The typical way to look for problems is with retrospective analysis of journal articles and case reports of unusual incidents or complications. Some of these problems are rapid joint degeneration, called chondrolysis, often blamed on chondrotoxic local anesthetic or exposure of the joint to excessive heat from RF devices. Others are the correlation of joint disease with other factors such as smoking or diabetes. No method exists today to directly study joint health over large populations.

During an arthroscopic procedure, the joint is inflated with fluid and tissue is removed with mechanical cutting devices such as an arthroscopic shaver or RF ablation device. This joint tissue, consisting of cartilage, blood, synovium, meniscus, and other tissues, is flushed out of the joint into a waste collection bucket and poured down the drain.

### SUMMARY

The invention described below is a device, system, and method for collecting previously discarded blood, cartilage, synovium, bone, meniscus and other tissue samples, collecting them in a dedicated collection cassette, pre-treating the samples as needed, transporting these samples to a lab, analyzing these samples, and aggregating this data into a searchable and structured dataset that may be analyzed, for correlation and causality of joint diseases and pathology. Tissue may be collected with devices and systems for use in an arthroscopic procedure, an open procedure such as a joint implant, or with a dedicated biopsy device.

Currently these tissue samples are being disposed of as medical waste. The present invention collects this biologic material, processes it, subjects it to a series of tests including biological assays and genetic sequencing, aggregates the data and imparts significant data value to what is now just flushed down the drain. The tissue samples are collected and prepared in a consistent way in order to get consistent and valid results.

Many genetic sequencing and testing devices rely on tiny amounts of material as they are normally obtained only by an invasive dedicated biopsy or blood test. An advantage of the present system and method is the ability to obtain sufficient quantities of joint connective tissue to be able to run robust DNA, RNA, and genetic tests.

Trend data indicates that by 2020 there will be 14 million arthroscopies around the world. This amounts to 14 million possible data points to measure the health and disease of joints around the world, and across ethnic, gender, and phenotype groups. This database will be an exceptionally valuable resource to treat individual patients as well as at-risk patient populations, and to structure preventative care and policies to prevent joint disease.

### BRIEF DESCRIPTION OF THE DRAWINGS

FIG. 1 is an overview of tissue gathering in an arthroscopic procedure.

FIG. 2 is a flow diagram of collection of arthroscopic surgery tissue material and preparation for testing.

FIG. 3 is an overview of tissue gathering in an open surgical procedure.

FIG. 4 is a flow diagram of collection of tissue material from an open surgery and preparation for testing.

FIG. 5 is a device for collecting and separating tissue samples.

FIG. 6 is a flow diagram of transportation of data to a lab for testing and collating of the aggregated data for storage and analysis.

### DETAILED DESCRIPTION OF THE INVENTIONS

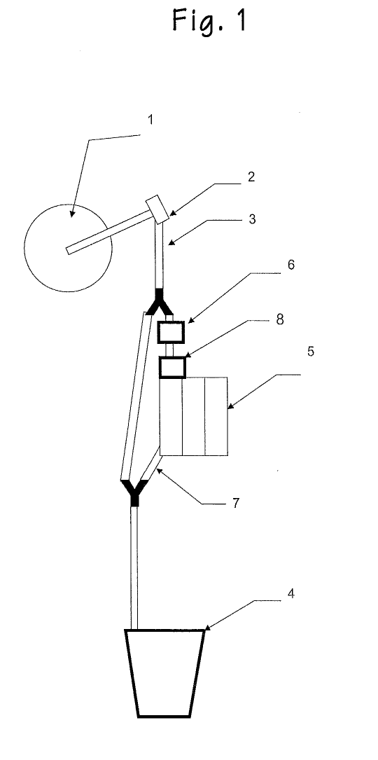

FIG. 1 illustrates a system for collecting joint tissue of various types from a joint space in an arthroscopic surgical procedure into a collection cassette for data analysis. As shown in FIG. 1, during an arthroscopic procedure, the joint space 1 is inflated with fluid and tissue is removed with a surgical device 2 such as an arthroscopic shaver, RF ablation device, or other mechanical cutting device. This joint tissue, consisting of cartilage, tendon, bone, muscle, blood, synovium, meniscus shavings, and other tissues, is flushed out of the joint, generally through a drainage tube 3 and into a waste collection bucket 4. In line with the drainage tube and the waste collection bucket is a collection cassette 5. The collection cassette is connected to the drain line of an arthroscopic device such as an arthroscope, a shaver, cannula, or a dedicated biopsy device that has a fluid drain from the joint. The collection cassette collects, separates, and preps the joint tissue (cartilage, tendon, bone, muscle, blood, synovium, meniscus shavings, etc.) from inside the joint into the cassette 5.

Upstream of the cassette, or integrated into it are a temperature and pressure activated valve 6. When a pressure sensor senses that the cassette is full, the diverter valve diverts the outflow through a bypass 7 to the waste bucket 4. Similarly, when a temperature sensor senses that the temperature of the outflow fluid goes over a preset limit (e.g. 45 degrees Celsius) the diverter valve 6 directs fluid away from the cassette so as not to spoil the sample with heat, e.g. the heated fluid produced by a radio frequency ablation or cutting device.

Inline with the system, there may be a pre-processor (e.g. a grinding device) 8 to make the tissue samples of a consistent particle size. This may be in-line or integral with the cassette, or the outflow device from the joint, such as the shaver, arthroscopy, cannula, or dedicated biopsy device. These particles may be sorted in the cassette through microfluidic channels, or may be separated in a centrifuge either in the operating room or later in the lab.

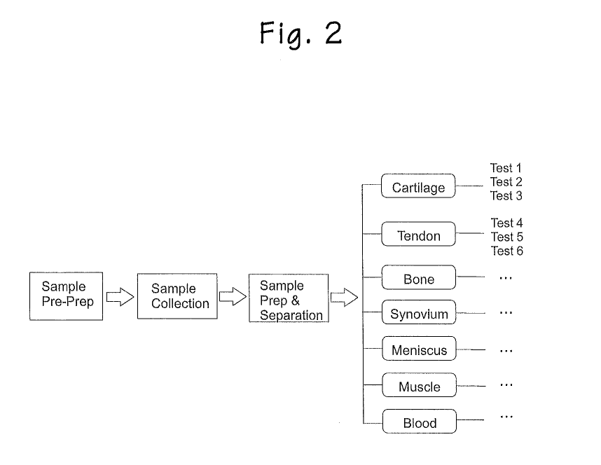

FIG. 2 details how the system collects and separates biological samples from joint tissue surgical effluent. The tissue types are pre-prepped, separated, and collected, and each tissue type can have a number of tests run for pathogens, gene mutations, protein expression, effect of heat, effect of fluid pressure, and effect of anesthetics on joint health. Tests can also be run to detect across a population the breakdown products of absorbable implants and sutures, and metal ions, and if these are causing degenerative changes in the joint.

The numbers of possible conditions that the system can uncover are large, depending on the number of tissue types (seven here as shown) and the number of tests run on each sample. This system can query a population of patients and uncover preventable joint diseases, or can query the data pool as to the effectiveness of treatments, or the side effects of treatments or to aid in discovering targets for new drug development.

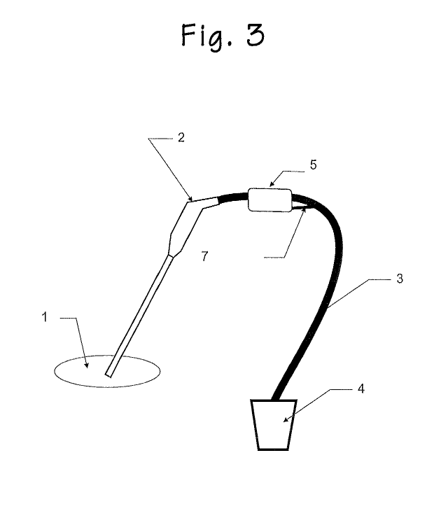

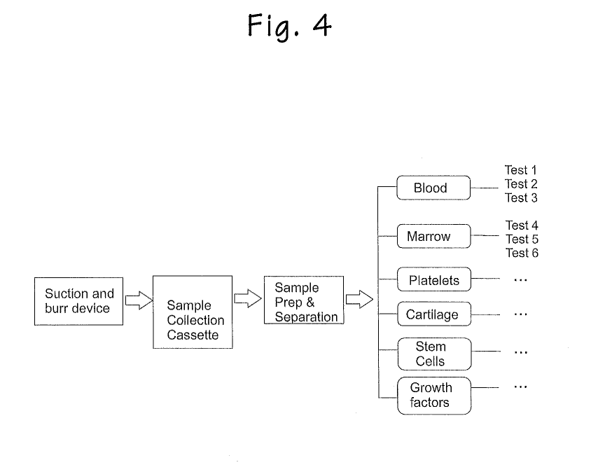

FIGS. 3 and 4 illustrate a system for collecting blood and tissue from a surgical site 1 in traditional open orthopedic surgeries such as hip and knee implants. These open surgeries may generate biologic tissue such as bone marrow, blood, bone, platelets, stem cells, growth factors, and cartilage material. This can be sampled with a surgical device 2 with suction and an optional cutting burr that can also shave off samples of bone and cartilage hard tissue that are being resected, for example the femoral head that is cut off during a hip implant, or the parts of the femoral condyle cut off when doing a knee implant. An in-line collection cassette 5 collects the tissue constituents. A vent or bypass system 7 ensures that the drain line remains free flowing when the collection cassette is full, wherein once the collection cassette is full, the surgical effluent bypasses the collection cassette and is suctioned through the suction line 3 into the waste bucket 4. A pre-processor 8 (e.g. a grinder), as shown in FIG. 1, may also be included in this system.

A means 5 for separating the tissue constituents into blood, bone marrow, stem cells, cartilage, etc. collates the tissue. The separated tissue types can each have a number of tests run on the tissue sample. These tissue samples are tested, and data patterns between the test results are developed using various data analysis techniques. All tissue contains data, and the combinations and permutations of these test data sets can yield significant insights that cannot be gained otherwise. Methods to structure and analyze this data can include: A/B testing, association rule learning, classification, cluster analysis, data fusion and integration, machine learning, neural networks, pattern recognition, predictive modeling and data visualization.

Predictive algorithms can analyze the data set to predict the risk of implant loosening or other complications (post operative healing of soft tissue repairs, absorption of suture and tissue anchors, and prevention of costly complications) based on comparing the sample to others in the data set. This will be especially valuable for post market surveillance of implants and to predict who may need implants, and to help predict the useful life of the implant in a particular patient. The collection and analysis of the tissue types specific to joints is especially valuable as this data incorporates the major areas of interest in healthcare data such as R&D data, clinical data, as well as patient activity and behavior data. These data will be valuable additions to the patient's EHR (electronic health record). These data are especially useful for comparative effectiveness research, discovery of new drugs and therapies, outcome tracking, and clinical and economic performance of joint surgery procedures. Having population scale data will greatly assist in discovering the mechanisms of action (MOA) of popular therapies such as PRP injections (platelet rich plasma) where the exact MOA is controversial and efficacy is inconsistent.

FIGS. 3 and 4 show the system for collecting tissue during an open surgery, such as with hip and knee implants. A suctioning device 2 suctions biologic tissue during the open orthopedic surgical procedure. The suctioning device may have an optional cutting burr if collection of hard tissue like bone and cartilage is desired.

Alternatively from the devices shown in FIGS. 1 and 3, the device that removes tissue from the joint may be a dedicated biopsy device, and may be sufficiently small in diameter (e.g. 3 mm) that it would allow for collection of tissue samples in an outpatient rather than an inter-operative operating room setting. This biopsy collection may be combined with a diagnostic arthroscopy procedure.

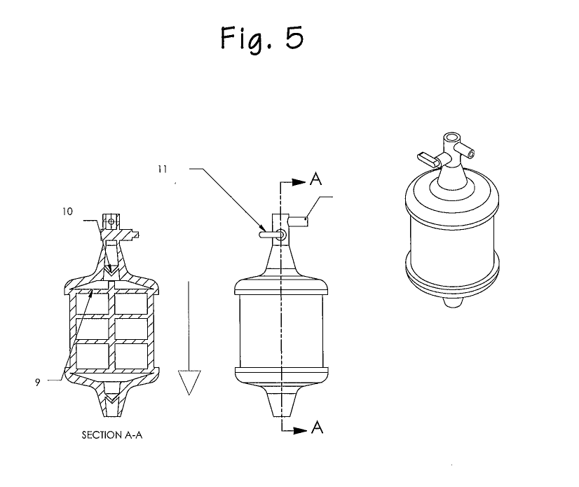

One possible device for collecting and separating tissue samples is shown in FIG. 5. The medical effluent enters through a one-way valve 10. Progressively finer screens or filters 9 trap the different types of biologic material, such as bone, cartilage, meniscus, and blood. The various filters 9 are stacked, starting with a coarse filter for bone, a finer filter for cartilage, finer still for meniscus, and the finest filter for blood. The collection filter stack collects the biologic tissue. A bypass valve 11 stops tissue collection when the filters are full and vents drainage to the waste bucket. The filter stack is on a spindle and removable and separable for later analysis of the biologic tissues. The cartridge may also have sections with reagents that selectively bind to specific tissue types for targeted tests.

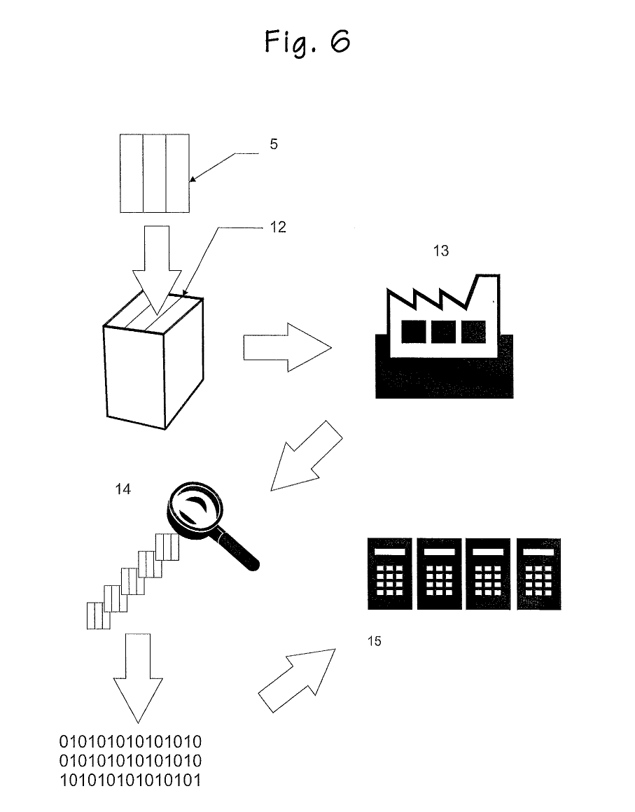

FIG. 6 is a flow diagram of transportation of data to a lab for lab testing and collating of the aggregated data for storage and analysis. Once the samples have been collected, whether in an arthroscopic procedure, an open procedure, or with a dedicated biopsy device, the cassette prepared samples 5 may be preserved with reagents packaged with the cassette, such as

PrepProtect (Miltenyi Biotec, San Diego) or PaxGene (Qiagen, Hilden GR), or the cassette may be placed in a cooled storage and shipping container 12. These samples are then sent to a lab 13 for analysis. Analysis may consist of genetic sequencing with rapid sequencing devices such as those made by Illumina (San Diego Calif.).

At the lab, a panel of tests 14 are run on the joint tissue samples. These include analysis of the blood, cartilage, synovium, bone and meniscus samples and tests for health and pathology in these samples. The tests are done in a structured and consistent manner for later data analysis.

The lab tests are aggregated 15, and the patient data is encrypted. This encryption is done to comply with HIPAA standards such as: “Encryption and Decryption”—164.312 and “Encryption”—164.312. The results of the tests may be shared with the patient if they request the results. The tissue collection and tests are done under patient informed consent.

The surgical record is included with the sample in a metafile. The surgical operating conditions (pressure and temperature of fluid) are included and saved with the sample as a metafile. A record of drugs taken by the patient and any local anesthesia is collected and included in a metafile. The tissue is sent for a battery of tests, and for genetic sequencing. The large data set is then mined to develop preventative treatments and lifestyle modifications to prevent joint degenerative disease such as arthritis, or ligament and meniscal tears. The large data set can be used with an artificial intelligence (AI) engine to suggest treatment algorithms for patients.

The system relies on rapid genomic sequencing, such as used by 23andMe® (Mountain View, Calif.), but rather than general genetic information derived from saliva, the system uses testing and genotyping and phenotyping specific to joint tissue and joint health. This system can give a revolutionary new window into joint health and disease prevention, rather than treating diseased joints when symptoms appear (once symptoms it is often too late to stop or reverse the damage to the joints). This system can be a powerful tool to identify risk factors for disease and prevent the degeneration in the first place. Other risk factors can be identified, such as predispositions to pulmonary embolism during arthroscopy (a feared and fatal complication), as well as sensitivity to the cartilage-killing effects of some local anesthetics like Marcaine™, commonly used in arthroscopic surgery and post op care. The larger the data set, the more effective and valuable it becomes in identifying risk factors for joint diseases and for developing strategies to prevent them.

It is highly desirable for a system to exist that captures some of this tissue, runs a series of tests on it for the benefit of the patient, and then this data, with the patient identity encrypted and removed, is aggregated into a large population database, where data analysis can be performed. These tests would look for correlation between such things as joint health and diabetes, the effect of age on joint health, relationships between physical or genetic conditions, and search for previously hidden causes of degenerative diseases such as osteoarthritis. New opportunities to study the metagenomes, proteomes, metabolomes, and metatrascriptomes of the joint, will now be possible. Factors such as pH levels and inflammatory cytokines can be studied longitudinally. Communities of disease can be identified in a way not now possible. Also communities of robust joint health will be identified, as well as the variables that contribute to health can be studied and potentially duplicated. Data can also be compiled to study, treat, and prevent diseases such as bone and synovial cancers.

While the preferred embodiments of the devices and methods have been described in reference to the environment in which they were developed, they are merely illustrative of the principles of the inventions. The devices, systems and methods have been described with regard to surgical procedures in the area of orthopedic, joint and spine surgery, however the devices and methods may be used in any surgical procedure where medical waste is generated and traditionally discarded. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A method for capturing, collecting and processing tissue samples typically discarded as medical waste in an arthroscopic surgical procedure, analyzing these tissue samples to generate genetic health and disease information, and storing the test results in a searchable database, said method comprising the steps of:
inflating a joint space with fluid;
performing an arthroscopic surgical procedure;
suctioning biologic waste during the arthroscopic surgical procedure, said biologic waste comprising various types of joint tissue;
collecting the various types of joint tissue into a collection means;
separating the various types of joint tissue into separate tissue type samples;
prepping the tissue type samples;
testing the tissue type samples against various test criteria and generating test results;
storing the test results in a searchable and structured database; and
analyzing the database to discover correlation and causality of joint diseases and pathology.

2. The method of claim 1 wherein the testing tissue type samples further comprises biological assays.

3. The method of claim 1 wherein the testing tissue type samples further comprises genetic sequencing.

4. The method of claim 1 further comprising storing information regarding the surgical operating conditions, record of drugs taken by the patient, and local anesthesia given to the patient with the tissue type samples.
